(19) United States
(12) Patent Application Publication
(10) Pub. No.: US 2024/0025808 A1
(43) Pub. Date: Jan. 25, 2024

(54) A GLASS IONOMER CEMENT

(71) Applicant: UNIVERSITY OF LIMERICK, Limerick (IE)

(72) Inventors: Eamonn DE BARRA, Limerick (IE); Robert G HILL, Maidenhead (GB); Ian HOULIHAN, Limerick (IE); Aisling FEEN, Clonakitty (IE); Ken MURPHY, Athy (IE)

(21) Appl. No.: 18/245,591

(22) PCT Filed: Sep. 16, 2021
(86) PCT No.: PCT/EP2021/075528

§ 371 Date: Mar. 16, 2023

(30) Foreign Application Priority Data
Sep. 17, 2020 (GB) 2014632.0

(51) Int. Cl. C04B 28/28; C04B 14/22

(52) U.S. Cl. CPC C04B 28/28; C04B 14/22; C04B 2201/50; C04B 2103/408

(57) ABSTRACT

There is provided a two part hardenable composition comprising a powder first part and a liquid second part. The parts are operable to form a glass ionomer cement which hardens to a solid mass upon mixing of the parts together. The composition comprises an inorganic glass and/or salt in the powder first part, an aqueous carrier in the liquid second part and an acid-functional polymer in the powder first part and/or the liquid second part. The composition also comprises an at least partially water-miscible additive in the first and/or second parts. The additive is operable to extend the working time of the glass ionomer cement upon mixing of the first and or second parts. The composition is operable to form a glass ionomer cement which, once hardened to a solid mass, has a plane strain fracture toughness of at least 0.7 MPa·m^{1/2} as measured at 24 hours from mixing. Also provided is a method of producing a glass ionomer cement.

### FIELD

The present invention relates to a two part hardenable composition comprising a powder first part and a liquid second part which are operable to form a glass ionomer cement which hardens to a solid mass upon mixing of the parts together. The present invention also extends to the two part hardenable compositions for use in the treatment of human or animal tooth and/or bone, a method of producing a glass ionomer cement from the two part hardenable compositions and a solid glass ionomer cement produced from the two part hardenable compositions.

### BACKGROUND

Glass ionomer cements (GICs), which are typically formed from two part hardenable compositions, are currently used in both orthopaedic and dental fields.

Glass ionomer cements are typically formed by mixing an ion leachable glass and an aqueous polymer solution of a polyelectrolyte or polyacid. The conventional setting reaction of a glass ionomer cement involves the carboxylic acid groups of the polymer hydrolysing and degrading the glass, releasing metal cations from the glass particles. These cations then chelate and crosslink with the COO— groups on the polyelectrolyte or polycarboxylic acid polymer backbone. This crosslinking mechanism creates a polysalt matrix in which the depleted glass particles are embedded.

Glass ionomer cements have advantages, such as, being water-based, having the ability to chemically bond to both dentin and bone, producing no significant exotherm upon setting and being bioactive (due to the release of therapeutic ions), for example. However, glass ionomer cements are typically non-load bearing and have inferior mechanical properties compared to other known cements. For example, glass ionomer cements are typically brittle and have poor mechanical strength, particularly, in terms of toughness and fracture toughness. This has restricted their use in load bearing applications in both dental and orthopaedic fields.

Glass ionomer cements also have the disadvantage that they typically have short working times. This means that an operator only has a relatively short period of time in which to insert and manipulate the cement into a bone or dental cavity, for example.

There are also formulation-based constraints on glass ionomer cements, which limit the handling and placement of such cements. For example, there are constraints on the glass volume fraction (GVf), polymer concentration, polymer molar mass and glass reactivity which can typically be used. By way of example, the use of high molar mass polymers gives a paste viscosity that is too high for a homogeneous cement mix to be formed. This renders such cements unsuitable for use in class I dental restoration, for example.

Attempts to improve the mechanical properties of glass ionomer cements include modifying the polyacid and the use of polyacids having a narrow molecular weight distribution. For example, WO 2010/102786 describes glass ionomer cements containing thiol groups, preferably on the polyacid. The thiol groups react with unsaturated carbon-carbon bonds in the system via and an ene or Michael addition reaction, for example, to provide additional crosslinking. The effect of the presence of the thiol groups on the rheological properties and/or the working time of the cement is not discussed.

Other attempts to improve the mechanical properties of glass ionomer cements include the use of resin modified glass ionomer cements (RM-GICs). In RM-GICs, some of the water in a conventional glass ionomer cement is replaced by a monomer that is typically capable of undergoing a free-radical polymerisation reaction (during curing).

WO 2016/202744 describes a glass ionomer cement comprising, among others, a polymer comprising acid groups and hydrolysis-stable pendant group having one or more polymerizable carbon-carbon double bonds and a hydrolysis-stable, water-soluble monomer having one polymerizable double bond. Additional crosslinking is provided by the free-radically initiated reaction of the polymerizable carbon-carbon double bonds.

It is an object of aspects of the present invention to provide one or more solutions to the above mentioned or other problems.

### SUMMARY

According to a first aspect of the present invention there is provided a two part hardenable composition comprising a powder first part and a liquid second part, the parts being operable to form a glass ionomer cement which hardens to a solid mass upon mixing of the parts together, the composition comprising an inorganic glass and/or salt in the powder first part, an aqueous carrier in the liquid second part and an acid-functional polymer in the powder first part and/or the liquid second part;

wherein the composition further comprises an at least partially water-miscible additive in the first and/or second parts, the additive being operable to extend the working time of the glass ionomer cement upon mixing of the first and or second parts;

and wherein the composition is operable to form a glass ionomer cement which, once hardened to a solid mass, has a plane strain fracture toughness of at least 0.7 MPa·m 112 as measured at 24 hours from mixing.

According to a second aspect of the present invention there is provided a method of producing a glass ionomer cement from a two part hardenable composition according to the first aspect of the present invention, the method comprising the step of mixing the powder first part and liquid second part.

According to a third aspect of the present invention there is provided a solid glass ionomer cement produced from mixing the powder first part and liquid second part of the two part hardenable composition according to the first aspect of the present invention.

According to a fourth aspect of the present invention there is provided a two part hardenable composition according to the first aspect of the present invention for use in the treatment of human or animal bone.

According to a fifth aspect of the present invention there is provided a two part hardenable composition according to the first aspect of the present invention for use for us in dentistry.

### DETAILED DESCRIPTION

For the avoidance of doubt, in the context of the present invention, the two part hardenable compositions become a glass ionomer cement upon mixing of the first and second parts thereof together. Once the glass ionomer cement has hardened to a solid mass the glass ionomer cement may be referred to herein as a ‘hardened glass ionomer cement’.

The two part compositions comprises a powder first part.

For the avoidance of doubt, by ‘powder’, and like terms as used herein, is meant a material in a solid, particulate form and is generally a free flowing dry particulate material usually made up of one or a mixture of powder(s) and not including a liquid carrier. The term “solid” means a non-liquid or non-gaseous material.

The powder first part comprises an inorganic glass and/or salt. Suitably, the inorganic glass and/or salt may comprise one or more elements that are capable of coordinating with the acid-functional polymer (i.e. with the acid groups of the acid-functional polymer). Thus, suitably, the inorganic glass and/or salt may comprise one or more cations that are capable of coordinating with the acid-functional polymer (i.e. with the acid groups of the acid-functional polymer). Suitably, the one or more elements, such as cations, that are capable of coordinating with the acid-functional polymer are leachable from the basic glass.

The inorganic salt and/or glass may be any suitable compound. Examples of suitable compounds include, but are not limited to, comprise tricalcium aluminate and/or a basic glass. Thus, the inorganic salt and/or glass may comprise tricalcium aluminate and/or a basic glass.

Suitably, the inorganic salt and/or glass may comprise a basic glass. A basic glass is a metal oxide or hydroxide, mineral silicate or ion leachable glass or ceramic that is capable of reacting with an polyelectrolyte in the presence of water to form a hydrogel.

Suitably, the basic glass may comprise one or more elements that are capable of coordinating with the acid-functional polymer (i.e. with the acid groups of the acid-functional polymer). Thus, suitably, the basic glass may comprise one or more cations that are capable of coordinating with the acid-functional polymer (i.e. with the acid groups of the acid-functional polymer). Suitably, the one or more elements, such as cations, that are capable of coordinating with the acid-functional polymer are leachable from the basic glass.

The basic glass may be any suitable basic glass. Suitable basic glasses will be known to a person skilled in the art. For example, the basic glass may comprise mixed metal oxides of calcium (Ca), barium (Ba), magnesium (Mg), strontium (Sr), aluminium (Al), silicon (Si), zinc (Zn), sodium (Na), potassium (K), boron (B), silver (Ag), titamium (Ti), yttrium (Y) and/or phosphorous (P). For example, the basic glass may comprise Ca/AI oxides and/or Sr/AI oxides. Suitably, the basic glass may comprise fluoride ions, such as leachable fluoride ions.

Suitably, the basic glass may comprise silicate glass, fluorosilicate glass, calcium silicate glass, calcium fluorosilicate glass, fluoroborosilicate glass, calcium fluoroborosilicate glass, strontium silicate glass, strontium fluorosilicate glass, strontium fluoroborosilicate glass, aluminosilicate glass, alumino fluorosilicate glass, calcium aluminosilicate glass, calcium alumino fluorosilicate glass, aluminium fluoroborosilicate glass, calcium aluminium fluoroborosilicate glass, strontium aluminosilicate glass, strontium aluminofluorosilicate glass, strontium aluminofluoroborosilicate glass, zinc silicate glass, zinc fluorosilicate glass, zinc fluoroborosilicate glass, zinc aluminosilicate glass, zinc alumino fluorosilicate glass, zinc aluminium fluoroborosilicate glass, titanium silicate glass, titanium fluorosilicate glass, titanium fluoroborosilicate glass, titanium aluminosilicate glass, titanium alumino fluorosilicate glass, titanium aluminium fluoroborosilicate glass, yttrium silicate glass, yttrium fluorosilicate glass, yttrium fluoroborosilicate glass, yttrium aluminosilicate glass, yttrium alumino fluorosilicate glass, yttrium aluminium fluoroborosilicate glass, chloride glasses, phosphate glasses and combinations thereof.

Further examples of basic glasses include, but are not limited to, metal oxides, such as zinc oxide and magnesium oxide and ion-leachable glasses. Suitable ion-leachable glass will be well known to a person skilled in the art.

Suitably, the basic glass may comprise a barium and/or strontium aluminofluorosilicate glass.

The surface of the basic glass particles may optionally be organically modified. For example, the surface of the basis glass particles may be silanized. In this case, the surface of the basic glass particles are typically modified with an organic silane compound such as, for example, vinyltrimethoxysilane, methacryloxypropyltrialkoxysilane, γ-aminopropyltrialkoxysilane, propyl trialkoxysilane and combinations thereof. Examples of other surface modifiers include, but are not limited to, include fatty acids, organic acids, surfactants, inorganic acids, polysiloxanes and combinations thereof. By such a treatment the compatibility of the basic glass particles with the other components of the composition may be improved. It will be appreciated by a person skilled in the art that organic modification of the surface of the basic glass particles is only suitable if the release of the reactive elements is not impeded.

The basic glass may be prepared by known methods. Suitable methods will be known to a person skilled in the art.

The basic glass may comprise a bioactive glass. The bioactive glass may be included in combination with conventional ionomer glasses. Examples of suitable bioactive glasses include, but are not limited to, those sold under the tradename Bioglass (RTM), such as Bioglass 45S5 (composition: 45 wt % SiO₂, 24.5 wt % Na₂O, 24.5 wt % CaO and 6 wt % P₂O₅), Bioglass BAG 58S, Bioglass BAG S53P4 and/or Bioglass BAG 70S 30C, fluoride containing bio active glasses, low sodium containing bioactive glasses and combinations thereof.

The basic glass may have any suitable average particle size. Suitably, the basic glass may have an average particle size up to 100 microns (μm), such a up to 75 μm, such as up to 50 μm, such as up to 40 μm, such as up to 25 μm, or even up to 10 μm. The basic glass may have an average particle size of at least 0.005 μm, such as at least 0.01 μm, such as at least 0.1 μm, such as at least 0.5 μm, or even at least 1 μm.

Suitably, the basis glass may have an average particle size of up to 40 μm.

The average particle size of the basic glass may be measured, for example, by electron microscopy or by using a conventional laser diffraction particle sizing method using a MALVERN Mastersizer S or MALVERN Mastersizer 3000 apparatus. Suitably, the average particle size of the basic glass may be measured using a conventional laser diffraction particle sizing method using a MALVERN Mastersizer S or MALVERN Mastersizer 3000 apparatus.

The basic glass may be present in any suitable amount. The two part hardenable composition my comprise at least 40 wt %, such as at least 50 wt %, such as at least 55 wt %, such as at least 60 wt %, such as at least 65 wt %, such as at least 70 wt %, such as at least 75 wt %, or even at least 80 wt % basic glass based on the total weight of the powder first part. The two part hardenable composition may comprise up to 100 wt %, such as up to 99.9 wt %, such as up to 95 wt %, such as up to 90 wt %, or even up to 85 wt % basic glass based on the total weight of the powder first part.

The two part hardenable composition may comprise from 40 to 100 wt %, such as from 50 to 100 wt %, such as from 55 to 100 wt %, such as from 60 to 100 wt %, such as from 65 to 100 wt %, such as from 70 to 100 wt %, such as from 75 to 100 wt %, or even from 80 to 100 wt % basic glass based on the total weight of the powder first part. The two part hardenable composition may comprise from 40 to 99.9 wt %, such as from 50 to 99.9 wt %, such as from 55 to 99.9 wt %, such as from 60 to 99.9 wt %, such as from 65 to 99.9 wt %, such as from 70 to 99.9 wt %, such as from 75 to 99.9 wt %, or even from 80 to 99.9 wt % basic glass based on the total weight of the powder first part. The two part hardenable composition may comprise from 40 to 95 wt %, such as from 50 to 95 wt %, such as from 55 to 95 wt %, such as from 60 to 95 wt %, such as from 65 to 95 wt %, such as from 70 to 95 wt %, such as from 75 to 95 wt %, or even from 80 to 95 wt % basic glass based on the total weight of the powder first part. The two part hardenable composition may comprise from 40 to 90 wt %, such as from 50 to 90 wt %, such as from 55 to 90 wt %, such as from 60 to 90 wt %, such as from 65 to 90 wt %, such as from 70 to 90 wt %, such as from 75 to wt %, or even from 80 to 90 wt % basic glass based on the total weight of the powder first part. The two part hardenable composition may comprise from 40 to 85 wt %, such as from 50 to 85 wt %, such as from 55 to 85 wt %, such as from 60 to 85 wt %, such as from 65 to 85 wt %, such as from 70 to 85 wt %, such as from 75 to 85 wt %, or even from 80 to 85 wt % basic glass based on the total weight of the powder first part.

Suitably, the two part hardenable composition may comprise from 40 to 100 wt % basic glass based on the total weight of the powder first part.

The two part hardenable composition may have any suitable glass volume fraction (GVf; also known as glass volume fraction ratio). The glass volume fraction is given as the volume of the basic glass divided by the volume of all constituents in the two part hardenable composition. The two part hardenable cement may have a glass volume fraction of at least 30%, such as at least 35%, such as at least 40%, such at least 45%, or even at least 50%. The two part hardenable composition may have a glass volume fraction up to 75%, such as up to 70%, such as up to 65%, or even up to 60%.

The two part hardenable composition may have a glass volume fraction from 30 to 75%, such as from 35 to 75%, such as from 40 to 75%, such as from 45 to 75%, or even from 50 to 75%. The two part hardenable composition may have a glass volume fraction from 30 to 70%, such as from 35 to 70%, such as from 40 to 70%, such as from 45 to 70%, or even from 50 to 70%. The two part hardenable composition may have a glass volume fraction from 30 to 65%, such as from 35 to 65%, such as from 40 to 65%, such as from 45 to 65%, or even from 50 to 65%. The two part hardenable composition may have a glass volume fraction from 30 to 60%, such as from 35 to 60%, such as from 40 to 60%, such as from 45 to 60%, or even from 50 to 60%.

Suitably, the two part hardenable composition may have a glass volume fraction from 40 to 60%.

The two part compositions comprise a liquid second part.

The term “liquid” as used herein does not require definition because it is well understood by the skilled person. However, for the avoidance of doubt it also includes a flowable material having a liquid carrier such as a slurry, suspension, emulsion or paste.

The liquid second part comprises an aqueous carrier. The aqueous carrier comprises water. Suitably, the aqueous carrier comprises at least 80 vol %, such as 90 vol %, such as 95 vol %, such as 99 vol %, such as 99.5 vol %, such as at least 99.9 vol %, or even 100 vol % water based on the total volume of the aqueous carrier.

It will be appreciated by a person skilled in the art that the aqueous carrier may therefore comprise up to 20 vol %, such as 10 vol %, such as 5 vol %, such as up to 1 wt %, such as up to wt %, or even up to 0.1 wt % of a solvent other than water based on the total volume of the aqueous carrier. Suitable solvents include, but are not limited to, aliphatic hydrocarbons such as mineral spirits and high flash point naphtha; aromatic hydrocarbons such as benzene, toluene, xylene and solvent naphtha 100, 150, 200; those available from Exxon-Mobil Chemical Company under the SOLVESSO trade name; alcohols such as ethanol, n-propanol, isopropanol and n-butanol; ketones such as acetone, cyclohexanone, methylisobutyl ketone and methyl ethyl ketone; esters such as ethyl acetate, butyl acetate and n-hexyl acetate; glycols such as butyl glycol; glycol ethers such as methoxypropanol, ethylene glycol monomethyl ether and ethylene glycol monobutyl ether; and combinations thereof.

Suitably, the aqueous carrier may comprise 100 vol % water based on the total volume of the aqueous carrier.

Advantageously, the use of an aqueous carrier means that the aqueous two part hardenable compositions are hydrophilic. This means that the aqueous two part hardenable compositions may be suitable for use in the human and/or animal body because they are capable of both wetting and forming intimate contact at fine topographical scales with water containing natural substrates such as teeth and bone, for example. The presence of an aqueous carrier promotes intimate contact between the composition and the substrate and thus enables improved adhesion.

The liquid carrier may be present in any suitable amount. The two part hardenable composition may comprise at least 10 wt %, such as at least 20 wt %, such as at least 30 wt %, such as at least 40 wt %, such as at least 45 wt %, or even at least 50 wt % liquid carrier based on the total weight of the liquid second part. The two part hardenable composition may comprise up to 99.9 wt %, such as 95 wt %, such as 90 wt %, such as up to 85 wt %, such as up to 80 wt %, such as up to 75 wt %, such as up 70 wt %, such as up to 65 wt %, such as up to 60 wt %, or even up to wt % liquid carrier based on the total weight of the liquid second part.

The two part hardenable composition may comprise from 10 to 99.9 wt %, such as from 20 to 99.9 wt %, such as from 30 to 99.9 wt %, such as from 40 to 99.9 wt %, such as from 45 to 99.9 wt %, such as from 50 to 99.9 wt % liquid carrier based on the total weight of the liquid second part. The two part hardenable composition may comprise from 10 to 99 wt %, such as from 20 to 99 wt %, such as from 30 to 99 wt %, such as from 40 to 99 wt %, such as from 45 to 99 wt %, such as from 50 to 99 wt % liquid carrier based on the total weight of the liquid second part. The two part hardenable composition may comprise from 10 to 95 wt %, such as from 20 to 95 wt %, such as from 30 to 95 wt %, such as from 40 to 95 wt %, such as from 45 to 95 wt %, such as from 50 to wt % liquid carrier based on the total weight of the liquid second part. The two part hardenable composition may comprise from 10 to 90 wt %, such as from 20 to 90 wt %, such as from 30 to 90 wt %, such as from 40 to 90 wt %, such as from 45 to 90 wt %, such as from 50 to 90 wt % liquid carrier based on the total weight of the liquid second part. The two part hardenable composition may comprise from 10 to 85 wt %, such as from 20 to 85 wt %, such as from 30 to 85 wt %, such as from 40 to 85 wt %, such as from 45 to 85 wt %, such as from 50 to 85 wt % liquid carrier based on the total weight of the liquid second part. The two part hardenable composition may comprise from 10 to 80 wt %, such as from 20 to 80 wt %, such as from 30 to 80 wt %, such as from 40 to 80 wt %, such as from 45 to 80 wt %, such as from 50 to 80 wt % liquid carrier based on the total weight of the liquid second part. The two part hardenable composition may comprise from 10 to wt %, such as from 20 to 75 wt %, such as from 30 to 75 wt %, such as from 40 to 75 wt %, such as from 45 to 75 wt %, such as from 50 to 90 wt % liquid carrier based on the total weight of the liquid second part. The two part hardenable composition may comprise from 10 to 70 wt %, such as from 20 to 70 wt %, such as from 30 to 70 wt %, such as from 40 to 70 wt %, such as from 45 to wt %, such as from 50 to 90 wt % liquid carrier based on the total weight of the liquid second part. The two part hardenable composition may comprise from 10 to 65 wt %, such as from 20 to wt %, such as from 30 to 65 wt %, such as from 40 to 65 wt %, such as from 45 to 65 wt %, such as from 50 to 65 wt % liquid carrier based on the total weight of the liquid second part. The two part hardenable composition may comprise from 10 to 60 wt %, such as from 20 to 60 wt %, such as from 30 to 60 wt %, such as from 40 to 60 wt %, such as from 45 to 60 wt %, such as from 50 to wt % liquid carrier based on the total weight of the liquid second part. The two part hardenable composition may comprise from 10 to 55 wt %, such as from 20 to 55 wt %, such as from 30 to 55 wt %, such as from 40 to 55 wt %, such as from 45 to 55 wt %, such as from 50 to 55 wt % liquid carrier based on the total weight of the liquid second part.

Suitably, the two part hardenable composition may comprise from 45 to 55 wt % liquid carrier based on the total weight of the liquid second part.

Suitably, the two part hardenable composition may comprise from 50 to 55 wt % liquid carrier based on the total weight of the liquid second part.

The two part hardenable composition comprises an acid-functional polymer.

By “acid-functional”, and like terms as used herein, is meant a polymer having at least one pendant or terminal acid group, such as a carboxylic acid group.

The acid functional polymer may be any suitable acid-functional polymer. Suitable acid-functional polymers will be known to a person skilled in the art. Examples of suitable acid-functional polymers include, but are not limited to, polyvinyl phosphonic acid, polyglutamic acids such as, for example, poly-γ-glutamic acid, acid-functional polyacrylics such as, for example, homopolymers and/or copolymers of acrylic acid, methacrylic acid, chloromethacrylic acid, itaconic acid, maleic acid, fumaric acid, aconitic acid, mesaconic acid, glutaconic acid and/or citraconic acid; anhydrides of the aforementioned acids; and combinations thereof.

Suitably, the acid-functional polymer may comprise acrylic acid.

Thus, suitably, the acid-functional polymer may comprise a homopolymer and/or copolymer of acrylic acid.

Suitably, the acid-functional polymer may comprise a homopolymer of acrylic acid.

The acid-functional polymer may have any suitable acid number (AN; also known as acid value or ‘AV’). The acid-functional polymer may have an acid number of at least 10 mg KOH/g, such as at least 20 mg KOH/g, such as at least 30 mg KOH/g, such as at least 40 mg KOH/g, such as at least 50 mg KOH/g, such as at least 60 mg KOH/g, such as at least 70 mg KOH/g, such as at least 80 mg KOH/g, such as at least 90 mg KOH/g, such as at least 100 mg KOH/g, such as at least 110 mg KOH/g, such as at least 120 mg KOH/g, such as at least 130 mg KOH/g, such as at least 140 mg KOH/g, or even at least 150 mg KOH/g.

The acid number is suitably expressed on solids.

As reported herein, the acid number was determined by titration with 0.1N methanolic potassium hydroxide solution. The sample of polymer (0.1-3 grams depending on acid number) was weighed accurately (on a balance with accuracy to weigh in milligrams) into a conical flask and was then dissolved in 25 millilitres of a solvent mixture containing dichloromethane and ethanol (3/1 w/w) and a few drops of 0.1% solution bromo thymol blue indicator; using light heating and stirring as appropriate. The solution was then cooled to room temperature (20-30° C.) and the solution titrated with the potassium hydroxide solution. The resulting acid number is expressed in units of mg KOH/g and is calculated using the following equation:

Acid Number=(titre KOH solution (mis)×Molarity KOH solution×56.1)/Weight of solid sample (grams)

All values for acid number reported herein were measured in this way.

The acid-functional polymer may have any suitable number-average molecular weight (Mn). The acid-functional polymer may have an Mn of at least 500 Daltons (Da=g/mole), such as at least 1,000 Da, such as at least 1,500 Da, such as at least 2,000 Da, such as at least 3,000 Da, such as at least 4,000 Da, such as at least 5,000 Da, such as at least 10,000 Da, such as at least 15,000 Da, such as at least 20,000 Da, such as at least 25,000 Da, or even at least 50,000 Da. The acid-functional polymer may have an Mn up to 250,000 Da, such as up to 200,000 Da, such as up to 150,000 Da, or even up to 100,000 Da.

The acid-functional polymer may have an Mn from 500 to 250,000 Da, such as from 1,000 to 250,000 Da, such as from 1,500 to 250,000 Da, such as from 2,000 to 250,000 Da, such as from 3,000 to 250,000 Da, such as from 4,000 to 250,000 Da, such as from 5,000 to 250,000 Da, such as from 10,000 to 250,000 Da, such as from 15,000 to 250,000 Da, such as from 20,000 to 250,000 Da, such as from 25,000 to 250,000 Da, or even from 50,000 to 250,000 Da. The acid-functional polymer may have an Mn from 500 to 200,000 Da, such as from 1,000 to 200,000 Da, such as from 1,500 to 200,000 Da, such as from 2,000 to 200,000 Da, such as from 3,000 to 200,000 Da, such as from 4,000 to 200,000 Da, such as from 5,000 to 200,000 Da, such as from 10,000 to 200,000 Da, such as from 15,000 to 200,000 Da, such as from 20,000 to 200,000 Da, such as from 25,000 to 200,000 Da, or even from 50,000 to 200,000 Da. The acid-functional polymer may have an Mn from 500 to 150,000 Da, such as from 1,000 to 150,000 Da, such as from 1,500 to 150,000 Da, such as from 2,000 to 150,000 Da, such as from 3,000 to 150,000 Da, such as from 4,000 to 150,000 Da, such as from 5,000 to 150,000 Da, such as from 10,000 to 150,000 Da, such as from 15,000 to 150,000 Da, such as from 20,000 to 150,000 Da, such as from 25,000 to 150,000 Da, or even from 50,000 to 150,000 Da. The acid-functional polymer may have an Mn from 500 to 100,000 Da, such as from 1,000 to 100,000 Da, such as from 1,500 to 100,000 Da, such as from 2,000 to 100,000 Da, such as from 3,000 to 100,000 Da, such as from 4,000 to 100,000 Da, such as from 5,000 to 100,000 Da, such as from 10,000 to 100,000 Da, such as from 15,000 to 100,000 Da, such as from 20,000 to 100,000 Da, such as from 25,000 to 100,000 Da, or even from 50,000 to 100,000 Da.

Suitably, the acid-functional polymer may have an Mn from 10,000 to 150,000 Da.

As reported herein, the Mn was determined by gel permeation chromatography using a polyethylene glycol (PEG) standard according to ASTM D6579-11 (“Standard Practice for Molecular Weight Averages and Molecular Weight Distribution of Hydrocarbon, Rosin and Terpene Resins by Size Exclusion Chromatography”. Gilson 132 dr detector; 254 nm, solvent: 0.1 M phosphate buffer, retention time marker: ethylene glycol, sample concentration: 0.2%).

All values for Mn reported herein were measured in this way.

Advantageously, acid-functional polymers having a higher number-average molecular weight than would typically be expected can be used in the two part hardenable compositions of the present invention. For example, typically, the use of high molecular weight acid-functional polymers can typically be expected to result in two part compositions that were physically and/or rheologically constrained. That is, the increased viscosity of polyacid solutions of high molecular mass polyacids is typically a limiting factor in the ability to physically form an homogenous cement paste, resulting in incomplete wetting of all glass particles and/or premature setting of the cement mass. However, advantageously, the use of the additives according to the present invention may mean that two part compositions comprising high molecular weight acid-functional polymers can successfully be mixed (i.e. because they are not physically and/or rheologically constrained). Similarly, a conventional glass ionomer composition is typically rheologically constrained by the reactivity of the ionomer glass and by its volume fraction. Thus, advantageously, ionomer glasses of increased reactivity than would typically be expected, for a given glass volume fraction and a given polyacid molecular weight and given concentration, can be used, via the present invention, to form a homogenous paste that otherwise would be physically constrained. Advantageously, an ionomer glass of a given reactivity, in combination with a polyacid of a given molecular mass and concentration may, via the present invention, have an increased glass volume fraction compared to what would typically be expected (i.e. the glass ionomer cements of the present invention may have a glass volume fraction beyond that which would typically result in incomplete homogenisation and/or incomplete mixing of all components).

The acid-functional polymer may be substantially linear or may be slightly branched. The degree of branching may be measured by the polydispersity index of said acid-functional polymer. The polydispersity index is given by the ratio of Mw to Mn (Mw/Mn), wherein Mw is the weight-average molecular weight and Mn is the number average molecular weight.

Suitably, the acid-functional polymer may have a polydispersity index of up to 20, such as up to 10, such as up to 7, such as up to 5, such as up to 2, or even up to 1.5. Suitably, the acid-functional polymer may have a polydispersity index from 1 to 20, such as from 1 to 10, such as from 1 to 7, such as from 1 to 5, such as from 1 to 2, or even from 1 to 1.5. Suitably, the acid-functional polymer may have a polydispersity index from 2 to 20, such as from 2 to 10, such as from 2 to 7, such as from 2 to 5.

Suitably, the acid-functional polymer may have a polydispersity index from 1 to 10.

Suitably, the acid-functional polymer may have a polydispersity index from 2 to 7.

The acid-functional polymer may be present in the in the powder first part and/or the liquid second part. Suitably, the acid-functional polymer may be present in the liquid second part.

The acid-functional polymer may be present in any suitable amount. The two part hardenable composition may comprise at least 10 wt %, such as at least 15 wt %, such as at least wt %, such as at least 25 wt %, such as at least 30 wt %, such as at least 35 wt %, such as at least 40 wt %, or even at least 45 wt % acid-functional polymer based on the total weight of the liquid second part. The two part hardenable composition may comprise up to 90 wt %, such as up to 80 wt %, such as up to 70 wt %, such as up to 60 wt %, such as up to 55 wt %, or even up to wt % acid-functional polymer based on the total weight of the liquid second part.

The two part hardenable composition may comprise from 10 to 90 wt %, such as from 15 to wt %, such as from 20 to 90 wt %, such as from 25 to 90 wt %, such as from 30 to 90 wt %, such as from 35 to 90 wt %, such as from 40 to 90 wt %, or even from 45 to 90 wt % acid-functional polymer based on the total weight of the liquid second part.. The two part hardenable composition may comprise from 10 to 80 wt %, such as from 15 to 80 wt %, such as from 20 to 80 wt %, such as from 25 to 80 wt %, such as from 30 to 80 wt %, such as from 35 to 80 wt %, such as from 40 to wt %, or even from 45 to 80 wt % acid-functional polymer based on the total weight of the liquid second part. The two part hardenable composition may comprise from 10 to 70 wt %, such as from 15 to 70 wt %, such as from 20 to 70 wt %, such as from 25 to 70 wt %, such as from 30 to 70 wt %, such as from 35 to 70 wt %, such as from 40 to 70 wt %, or even from 45 to 70 wt % acid-functional polymer based on the total weight of the liquid second part. The two part hardenable composition may comprise from 10 to 60 wt %, such as from 15 to 60 wt %, such as from 20 to 60 wt %, such as from 25 to 60 wt %, such as from 30 to 60 wt %, such as from 35 to 60 wt %, such as from 40 to 60 wt %, or even from 45 to 60 wt % acid-functional polymer based on the total weight of the liquid second part. The two part hardenable composition may comprise from 10 to 55 wt %, such as from 15 to 55 wt %, such as from 20 to 55 wt %, such as from 25 to 55 wt %, such as from to 55 wt %, such as from 35 to 55 wt %, such as from 40 to 55 wt %, or even from 45 to 55 wt % acid-functional polymer based on the total weight of the liquid second part. The two part hardenable composition may comprise from 10 to 50 wt %, such as from 15 to 50 wt %, such as from 20 to 50 wt %, such as from 25 to 50 wt %, such as from 30 to 50 wt %, such as from 35 to 50 wt %, such as from 40 to 50 wt %, or even from 45 to 50 wt % acid-functional polymer based on the total weight of the liquid second part.

Suitably, the two part hardenable composition may comprise from 40 to 60 wt % acid-functional polymer based on the total weight of the liquid second part.

Suitably, the two part hardenable composition may comprise from 40 to 50 wt % acid-functional polymer based on the total weight of the liquid second part.

The composition comprises an at least partially water-miscible additive (herein referred to as ‘the additive’ or ‘additive’).

By “water-miscible”, and like terms as used herein, is meant that the additive is able to form a homogenous mixture with water. For the avoidance of doubt, by “at least partially water-miscible”, and like terms as used herein, is meant that at least a portion of the additive is able to form a homogenous mixture with water.

The additive is operable to extend the working time of the glass ionomer cement upon mixing of the first and or second parts. By ‘extend the working time’, and like terms as used herein, is meant that the working time of a glass ionomer cement according to the invention is increased compared to a glass ionomer cement that is formed from the same component parts but in the absence of the additive(s) as described herein. Suitably, the working time of a glass ionomer cement according to the invention is increased by at least 5%, such as at least 10%, such as at least 20%, such as at least 30%, such as at least 40%, such as at least 50%, such as at least 60%, such as at least 70%, such as at least 80%, such as at least 90%, or even at least 100% compared to a glass ionomer cement that is formed from the same component parts but in the absence of the additive(s) as described herein.

Suitably, the additive is operable to extend the working time of the glass ionomer cement upon mixing of the first and or second parts by introducing free volume into said glass ionomer cement. Without being bound by theory, this may be achieved by at least two actions; formation of a covalent linkage with one or more polymer chains of the acid-functional polymer (and could thus be described as an internal plasticiser) and/or dispersion within the polysalt matrix of the hardened glass ionomer cement (and could thus be described as an external plasticiser).

Thus, the additive may comprise an additive operable to form a covalent linkage with one or more polymer chains of the acid-functional polymer. An additive operable to form a covalent linkage with one or more polymer chains of the acid-functional polymer additive suitably comprises at least one functional group operable to react with at least one functional group, such as a carboxylic acid group, on the acid-functional polymer. Such additives will herein be referred to as ‘reactive additives’.

Suitably, the reactive additive may be operable to form a covalent linkage with at least two (2), such as three (3), four (4), or more polymer chains of the acid-functional polymer. For the avoidance of doubt, when a reactive additive is operable to form a covalent linkage with two (2) polymer chains of the acid-functional polymer, the additive may react with functional groups, such as carboxylic acid groups, on two separate polymer chains so as to form covalent linkages therewith or may react with functional groups, such as carboxylic acid groups, such as carboxylic acid groups, on the same polymer chain so as to form covalent linkages therewith. When a reactive additive is operable to form a covalent linkage with three (3), four (4) or more polymer chains of the acid-functional polymer, the additive may react with functional groups, such as carboxylic acid groups, on separate polymer chains so as to form covalent linkages therewith and/or may react with functional groups, such as carboxylic acid groups, such as carboxylic acid groups, on the same polymer chain so as to form covalent linkages therewith.

Suitably, the reactive additive may comprise at least one functional group operable to react with acid-functionality on the acid-functional polymer. Suitably, therefore, the reactive additive reacts with one or more acid groups on the acid-functional polymer via standard reaction mechanisms. Suitably, the reactive additive does not react with other functionality such as, for example, unsaturated carbon-carbon bonds. Thus, suitably, the reactive additive does not react via a free-radically initiated reaction mechanism, i.e. the reaction is not free-radically initiated. Thus, suitably, the reactive additive does not react via an ene reaction or a Michael addition reaction.

For example, the reactive additive may comprise at least one oxirane, amine, hydroxyl and/or carbodiimide group, preferably at least one oxirane, amine and/or carbodiimide group.

Suitably, the reactive additive may comprise at least two (2) oxirane, amine, hydroxyl and/or carbodiimide groups, preferably at least two (2) oxirane, amine and/or carbodiimide groups. For the avoidance of doubt, in this context, the reactive additive may comprise at least two (2) of the same functional group or may comprise a combination of different functional groups.

Suitably, the reactive additive may comprise two (2) oxirane, amine, hydroxyl and/or carbodiimide groups, preferably two (2) oxirane, amine and/or carbodiimide groups. For the avoidance of doubt, in this context, the reactive additive may comprise two (2) of the same functional group or may comprise a combination of two different functional groups.

Examples of suitable reactive additives include, but are not limited to, organosilane compounds comprising one or more glycidyl groups such as, for example, 3-glycidyloxypropyltriethoxysilane, 3-glycidyloxypropyltrimethoxysilane, 3-glycidyloxyethyltriethoxysilane, 3-glycidyloxymethyltriethoxysilane, 3-glycidyloxymethyltriethoxysilane, 3-glycidyloxymethyltrimethoxysilane; neopentyl glycol diglycidyl ether, glycerol diglycidyl ether, 1,4-butanediol diglycidyl ether, N-(3-dimethylaminopropyl)-N′-ethylcarbodiimide, 1,3-butadiene diepoxide, adipic acid dihydrazide, polyethylenimine, ethylenediamine, poly(ethylene glycol) dig lycidyl ether, polyproplene glycol diglycidyl ether, glutaraldehyde, any suitable molecule containing an epoxide group and combinations thereof.

Suitably, the reactive additive may comprise poly(ethylene glycol) diglycidyl ether, 1,4-butanediol diglycidyl ether or a combination thereof. For the avoidance of doubt, tartaric acid is not a reactive additive.

The additive may be operable to become dispersed within the polysalt matrix of the hardened glass ionomer cement. Such additives will herein be referred to as ‘dispersion additives’. Without being bound by theory, the dispersion additives may act as a plasticiser, dispersant and/or a humectant.

The dispersion additives may be physically bound to one or more component of the polysalt matrix of the hardened glass ionomer cement.

Alternatively and/or additionally, the dispersion additives may be leachable from the polysalt matrix of the hardened glass ionomer cement.

Examples of suitable dispersion additives include, but are not limited to, triethyl citrate, tributyl citrate, acetyl tributyl citrate, polysorbates, polyethylene glycol, propylene glycol, 1,2 epoxybutane, 1,2-epoxydodecane, 1,2-epoxypentane, 1,2-epoxytetradecane, glycidyl hexadecyl ether, glycidyl isopropyl ether, octyl glycidyl ether, decyl glycidyl ether and combinations thereof.

Suitably, the dispersion additive may comprise triethyl citrate, polysorbates or a combination thereof.

The additive may comprise one or more reactive additive and/or one or more dispersion additive.

Suitably, the additive may comprise at least one reactive additive.

Suitably, the additive may comprise at least one dispersion additive.

The additive may be present in any suitable amount. The two part hardenable composition may comprise at least 0.01 mole %, such as at least 0.05 mole %, such as at least 0.1 mole %, such as at least 0.2 mole %, such as at least 0.25 mole %, such as a least 0.5 mole %, such as at least 0.75 mole %, such as at least 1 mole %, such as at least 1.5 mole %, such as at least 2 mole %, or even at least 2.5 mole % additive based on the number of moles of the repeat (or monomer) unit of the acid-functional polymer. The two part hardenable composition may comprise up to 20 mole %, such as up to 15 mole %, such as up to 10 mole %, such as up to 7.5 mole %, such as up to 5 mole %, such as up to 4 mole %, or even up to 3 mole % additive based on the number of moles of the repeat (or monomer) unit of the acid-functional polymer.

The two part hardenable composition may comprise from 0.01 to 20 mole %, such as from 0.05 to 20 mole %, such as from 0.1 to 20 mole %, such as from 0.2 to 20 mole %, such as from 0.25 to 20 mole %, such as from 0.5 to 20 mole %, such as from 0.75 to 20 mole %, such as from 1 to 20 mole %, such as from 1.5 to 20 mole %, such as from 2 to 20 mole %, or even from 2.5 to 20 mole % additive based on the number of moles of the repeat (or monomer) unit of the acid-functional polymer. The two part hardenable composition may comprise from 0.01 to 15 mole %, such as from 0.05 to 15 mole %, such as from 0.1 to 15 mole %, such as from 0.2 to 15 mole %, such as from 0.25 to 15 mole %, such as from 0.5 to 15 mole %, such as from 0.75 to 15 mole %, such as from 1 to 15 mole %, such as from 1.5 to 15 mole %, such as from 2 to 15 mole %, or even from 2.5 to 15 mole % additive based on the number of moles of the repeat (or monomer) unit of the acid-functional polymer. The two part hardenable composition may comprise from 0.01 to 10 mole %, such as from 0.05 to 10 mole %, such as from 0.1 to 10 mole %, such as from 0.2 to 10 mole %, such as from 0.25 to 10 mole %, such as from 0.5 to 10 mole %, such as from 0.75 to 10 mole %, such as from 1 to 10 mole %, such as from 1.5 to 10 mole %, such as from 2 to 10 mole %, or even from 2.5 to 10 mole % additive based on the number of moles of the repeat (or monomer) unit of the acid-functional polymer. The two part hardenable composition may comprise from 0.01 to 7.5 mole %, such as from 0.05 to 7.5 mole %, such as from 0.1 to 7.5 mole %, such as from 0.2 to 7.5 mole %, such as from 0.25 to 7.5 mole %, such as from 0.5 to 7.5 mole %, such as from 0.75 to 7.5 mole %, such as from 1 to 7.5 mole %, such as from 1.5 to 7.5 mole %, such as from 2 to 7.5 mole %, or even from 2.5 to 7.5 mole % additive based on the number of moles of the repeat (or monomer) unit of the acid-functional polymer. The two part hardenable composition may comprise from 0.01 to 5 mole %, such as from 0.05 to 5 mole %, such as from 0.1 to 5 mole %, such as from 0.2 to 5 mole %, such as from 0.25 to 5 mole %, such as from 0.5 to 5 mole %, such as from 0.75 to 5 mole %, such as from 1 to 5 mole %, such as from 1.5 to 5 mole %, such as from 2 to 5 mole %, or even from 2.5 to 5 mole % additive based on the number of moles of the repeat (or monomer) unit of the acid-functional polymer. The two part hardenable composition may comprise from 0.01 to 4 mole %, such as from 0.05 to 4 mole %, such as from 0.1 to 4 mole %, such as from 0.2 to 4 mole %, such as from 0.25 to 4 mole %, such as from 0.5 to 4 mole %, such as from 0.75 to 4 mole %, such as from 1 to 4 mole %, such as from 1.5 to 4 mole %, such as from 2 to 4 mole %, or even from 2.5 to 4 mole % additive based on the number of moles of the repeat (or monomer) unit of the acid-functional polymer. The two part hardenable composition may comprise from 0.01 to 3 mole %, such as from 0.05 to 3 mole %, such as from 0.1 to 3 mole %, such as from 0.2 to 3 mole %, such as from 0.25 to 3 mole %, such as from 0.5 to 3 mole %, such as from 0.75 to 3 mole %, such as from 1 to 3 mole %, such as from 1.5 to 3 mole %, such as from 2 to 3 mole %, or even from 2.5 to 3 mole % additive based on the number of moles of the repeat (or monomer) unit of the acid-functional polymer.

The two part hardenable composition may comprise from 0.05 to 20 mole % additive based on the number of moles of the repeat (or monomer) unit of the acid-functional polymer.

The present inventors have surprisingly found that the glass ionomer cements of the present invention may provide a number of advantages during the working time of the cement, setting and/or in the final hardened glass ionomer cement.

Advantageously, the use of one or more additives, such as reactive additives and/or dispersion additives, may result in a glass ionomer cement having longer working times than would typically be expected. For example, the use of one or more additives, such as reactive additives and/or dispersion additives, may results in a glass ionomer cement that has a sufficiently low viscosity for longer than would typically be expected upon mixing of the parts together, meaning an operator has more time to insert and/or manipulate the glass ionomer cement prior to said glass ionomer cement setting. Without being bound by theory, this may occur by the introduction of free volume in the glass ionomer cement upon mixing of the parts together.

Advantageously, the use of one or more additives, such as reactive additives and/or dispersion additives, may result in glass ionomer cements having improved rheology upon mixing of the parts together than would typically be expected. This may mean that, for example, an increased glass volume fraction can be introduced, that basic glasses that have a higher reactivity than would typically be tolerated can be used, that acid-functional polymers having a higher molecular weight, such number-average molecular weight (Mn), than would typically be tolerated can be used and that a higher concentration of acid-functional polymer than would typically be tolerated can be used. Thus, advantageously, the use of one or more additives, such as reactive additives and/or dispersion additives, may make it possible to increase the limits of the glass volume fraction, glass reactivity, acid-functional polymer Mn and acid-functional polymer concentration beyond values that would typically be expected.

Advantageously, the use of one or more additives, such as reactive additives and/or dispersion additives, may result in a hardened glass ionomer cement having improved mechanical properties than would typically be expected. For example, the hardened glass ionomer cements of the present invention may advantageously have improved toughness and/or fracture toughness than would typically be expected. This means that the glass ionomer cements of the present invention may be used in a load bearing capacity, for example. It is a further advantage that the hardened glass ionomer cements of the present invention may have improved toughness and/or fracture toughness over a longer period of time than would typically be expected.

Typically, the magnitude of the effect of each of the aforementioned advantages is increased with the molecular mass of the additive, i.e for a given mole % of an additive, if its molecular mass is increased, the effect under observation is typically increased. Advantageously, this means that further widening of compositional limits can be achieved.

For the avoidance of doubt, the use of one or more additives, such as reactive additives and/or dispersion additives, may result in one or more of the above mentioned advantages.

The additive may be present in the powder first part and/or liquid second part. Suitably, the additive may be present in the part that ensures that the two part hardenable composition has a suitable shelf life, i.e. such that the two part hardenable composition is storage stable. By ‘storage stable’, and like terms as used herein, is meant that the liquid second part remains as a free flowing liquid, for example one having a viscosity between 10 and 10,000 centipoise for a period of at least 6 months, such as at least 12 months, or even at least 24 months under normally acceptable storage conditions of temperature, i.e. between 5 and 30° C. Accordingly, the liquid second part remains a free flowing liquid until mixed and/or activated with the powder first part as set out herein. Suitably, the additive may be present in the liquid second part.

The two part hardenable composition may optionally comprise one or more further components, such as components conventionally used in glass ionomer cements. Suitable components will be known to a person skilled in the art. Examples of suitable further components include, but are not limited to, chelating agents, curing accelerators, fillers, radiopacifying agents, pigments, antimicrobial agents, stabilisers, pH adjusters, ultraviolet absorbers, thickening agents, fluorescent agents and combinations thereof.

For example, the two part hardenable composition may optionally comprise a chelating agent. Suitable chelating agents will be known to a person skilled in the art. Examples of suitable chelating agents include, but are not limited to, tartaric acid.

The chelating agent, when present, may be present in the powder first part and/or the liquid second part.

Suitably, the chelating agent, when present, may be present in the liquid second part.

For example, the two part hardenable composition may optionally comprise a radiopacifying agent.

“Radiopacifying”, and like terms, as used herein means the ability to render a material more distinguishable from surrounding material when subjected to X-rays.

The two part hardenable composition may comprise any suitable radiopacifying agent. Suitable radiopacifying agents will be known to a person skilled in the art. Examples of suitable radiopacifying agents include, but are not limited to, zirconium dioxide, bismuth oxides, iodine compounds such as iohexal, iodixanol, strontium carbonate, powdered gold, powdered platinum, powdered iridium, powdered, powdered tantalum, powdered rhodium, powdered tungsten, barium sulphate, zirconium oxide and combinations thereof.

The radiopacifying agent, when present, may be present in the powder first part and/or the liquid second part.

It will be appreciated by a person skilled in the art that in the hardened glass ionomer cement the radiopacifying agent, when present, is suitably distributed throughout the polysalt matrix of said hardened glass ionomer cement.

For example, the two part hardenable composition may optionally comprise an antimicrobial agent.

The two part hardenable composition may comprise any suitable antimicrobial agent. Suitable antimicrobial agents will be known to a person skilled in the art. Examples of suitable antimicrobial agents include, but are not limited to, chlorhexidine, tetramycin, penicillin, keflex, ampicillin, cephalosporin, 12-(meth)acryloyloxydodecylpyridinium bromide, cetylpyridinium chloride, quaternary ammonium salts, epigallocatechin-3-gallate, triclosan and combinations thereof.

The antimicrobial agent, when present, may be present in the powder first part and/or the liquid second part.

Suitably, the two part hardenable compositions do not comprise a free-radical initiator.

Suitably, the two part hardenable compositions do not comprise a monomer component, such as an ethylenically unsaturated monomer component. Thus, suitably, the two part hardenable compositions are not resin modified glass ionomer cements (RM-GICs).

The powder first part and liquid second part are operable to form a glass ionomer cement which hardens to a solid mass upon mixing of the parts together.

Thus, according to a second aspect of the present invention there is provided a method of producing a glass ionomer cement from a two part hardenable composition according to the first aspect of the present invention, the method comprising the step of mixing the powder first part and liquid second part.

Suitable features of the second aspect of the present invention are as defined herein in relation to the first aspect of the present invention.

The glass ionomer cement may have any suitable working time. The working time is the time during which the viscosity of the cement is such that it may be manipulated by an operator. The glass ionomer cement may have a working time of at least 2 minutes (mins), 3 mins, such as at least 4 mins, such as at least 5 mins, such as at least 6 mins, such as at least 7 mins, such as at least 8 mins, such as at least 9 mins, such as at least 10 mins, such as at least 12 mins, such as at least 15 mins, such as at least 20 mins, or even at least 30 mins.

After the working time, i.e. once the glass ionomer cement has hardened to a solid mass, the glass ionomer cements can no longer be manipulated by an operator. The time in which is takes the glass ionomer cement to harden to a solid mass is known as the setting time. The glass ionomer cements may have any suitable setting time. The glass ionomer cements may suitably have a clinically appropriate setting time.

The mixing of the solid first part and storage stable liquid second part may be carried out by any suitable technique. For example, the mixing of the solid first part and storage stable liquid second part may be performed by a manual mixing process or by the use of an optionally adapted syringe or caulking gun.

A general procedure for mixing the powder first part and the liquid second part of the two part hardenable composition of the invention is described as follows: before mixing, the two parts are equilibrated for a suitable period, such as 1 hour or more at a temperature of 5 to 40° C., such as 10 to 35° C., or even 15 to 30° C. The powder first part is mixed with a suitable amount of liquid second part according to the ratios defined herein. Mixing is then carried out at the equilibrated temperature for at least 5 seconds, such as at least 20 seconds, or even at least 30 seconds. When the glass ionomer cement has been suitably mixed, the material may be injected or inserted within a cavity, such as a bone or dental cavity, and allowed to harden. The glass ionomer cement is suitably injected or inserted into a cavity, such as a bone or dental cavity, within the working time of the glass ionomer cement (as hereinbefore described).

Alternatively, the powder first part and liquid second part may be mixed at the equilibrated temperature in a static mixer connected to twin compartments of a capsule system which fits post mixing to a syringe or caulking gun, for example. When the material has been suitably mixed, the glass ionomer cement may be injected into a cavity, such as a bone or dental cavity, and allowed to harden.

Suitably, the mixing of the powder first part and liquid second part may be performed by the use of an optionally adapted syringe or caulking gun.

The powder first part and liquid second part may be mixed together at any suitable weight ratio. The powder first part and liquid second part may be mixed at a weight ratio from 10:1 to 1:2, such as from 8:1 to 1:1, such as from 6:1 to 1:1, such as from 5:1 to 1:1, such as from 4:1 to 1:1, such as from 3:1 to 2:1, such as from 2.5:1 to 2:1.

When the powder first part and liquid second part are mixed together, the carboxylic acid groups of the acid-functional polymer begin to hydrolyse and the basic glass begins to degrade, releasing metal cations (resulting in depleted glass particles). The cations then chelate and crosslink with the COO- groups on the acid-functional polymer creating a polysalt matrix in which the depleted glass particles are embedded. The mixture starts out at a relatively low viscosity and progresses to a stiffer and stiffer system that eventually hardens completely to a hardened glass ionomer cement.

Thus, according to a third aspect of the present invention there is provided a solid glass ionomer cement produced from mixing the powder first part and liquid second part of the two part hardenable composition according to the first aspect of the present invention.

Suitable features of the third aspect of the present invention are as defined herein in relation to the first and/or second aspects of the present invention.

The two part hardenable composition is operable to form a glass ionomer cement which, once hardened to a solid mass, has a plane strain fracture toughness of at least 0.7 MPa·m^{1/2} as measured at 24 hours. In other words, the hardened glass ionomer cement has a plane strain fracture toughness of at least 0.7 MPa·m^{1/2} as measured at 24 hours from mixing.

As reported herein, the plane strain fracture toughness, K_{Ic}, was determined by a double torsion (DT) fracture toughness test (de Barra E. and Hill R. “Influence of Alkali Metal Ions on the Fracture Properties of Glass Polyalkenoate Cements” Biomaterials 30 495-502(1998)). The DT specimens had the measurements 3 mm×65 mm×25 mm (t×I×w) and were produced using stainless steel moulds. The cement mixture was placed in these moulds and pressed between two plates using a G-clamp. A silicon-based mould release agent was used on the inner edges of the mould for ease of removal. They were then stored in a forced air oven pre-set to 37° C. ±2° C. for one hour. After this time, they were removed from the mould and placed in half litre containers filled with deionised water at 37° C.±2° C. until they were ready to be tested. For testing, a groove of 1 mm'1 mm was cut down the centre of the specimen as well as a sharp groove at the end of the specimen. Testing was carried out on a Universal Tensile Tester (H10KS Tinius Olsen) in a water bath at 37° C.±2° C. The specimen was loaded onto two parallel rollers 3 mm in diameter and spaced 20 mm apart. A constant crosshead speed of 0.1 mm/min was then applied to the end of the specimen which had the sharp groove with two 3 mm ball bearings spaced 10 mm apart (such that the loaded end of the specimen was subjected to a four-point bend). The plane strain fracture toughness, K_{Ic}, was calculated according to the following equation:

$${K}_{l c} = {P}_{C} {{W}_{m} ( \frac{3 ( 1 + v )}{W {t}^{3} {t}_{n}} )}^{\frac{1}{2}}$$

wherein P_{c} is the critical load required to fracture the specimen, Wm is the moment arm, W is the specimen width, t is the specimen thickness, to is the web thickness of the groove and v is Poisson's ratio (assumed to be 0.33).

All values for the plane strain fracture toughness reported herein were measured this way.

The hardened glass ionomer cement may have a plane strain fracture toughness of at least 0.75 MPa·m^{1/2}, such as at least 0.8 MPa·m ^{1/2}, such as at least 0.85 MPa·m^{1/2}, such as at least 0.9 MPa·m^{1/2}, such as at least 1 MPa·m^{1/2}, such as at least 1.05 MPa·m^{1/2}, such as at least 1.1 MPa·m^{1/2}, such as at least such as at least 1.15 MPa·m ^{1/2}, such as at least 1.2 MPa·m^{1/2}, such as at least 1.25 MPa·m^{1/2}, or even at least 1.3 MPa·m^{1/2} as measured at 24 hours from mixing.

The hardened glass ionomer cement may have a plane strain fracture toughness of at least MPa·m^{1/2} as measured at 24 hours from mixing.

The hardened glass ionomer cement may have any suitable plane strain fracture toughness as measured at 28 days from mixing. The hardened glass ionomer cement may have a plane strain fracture toughness of at least 0.7 MPa·m^{1/2} such as at least 0.75 MPa·m^{1/2}, such as at least MPa·m^{1/2}, such as at least 0.85 MPa·m^{1/2}, such as at least 0.9 MPa·m^{1/2}, such as at least 1 MPa·m^{1/2}, such as at least 1.05 MPa·m^{1/2}, such as at least 1.1 MPa·m^{1/2}, such as at least 1.15 MPa·m^{1/2}, such as at least 1.2 MPa·m^{1/2}, such as at least 1.25 MPa·m^{1/2}, or even at least 1.3 MPa·m^{1/2} as measured at 28 days from mixing.

The hardened glass ionomer cement may have a plane strain fracture toughness of at least MPa·m^{1/2} as measured at 28 days from mixing.

The hardened glass ionomer cement may have a plane strain fracture toughness of at least 1 MPa·m^{1/2} as measured at 28 days from mixing.

The hardened glass ionomer cement may have any suitable plane strain fracture toughness as measured at 84 days from mixing. The hardened glass ionomer cement may have a plane strain fracture toughness of at least 0.7 MPa·m ^{1/2}, such as at least 0.75 MPa·m ^{1/2}, such as at least MPa·m ^{1/2}, such as at least 0.85 MPa·m ^{1/2}, such as at least 0.9 MPa·m ^{1/2}, such as at least 0.95 MPa·m ^{1/2}, such as at least 1 MPa·m ^{1/2}, such as at least 1.05 MPa·m ^{1/2}, such as at least 1.1 MPa·m ^{1/2}, such as at least 1.15 MPa·m ^{1/2}, such as at least 1.2 MPa·m ^{1/2}, such as at least 1.25 MPa·m ^{1/2}, or even at least 1.3 MPa·m 112 as measured at 84 days from mixing.

The hardened glass ionomer cement may have a plane strain fracture toughness of at least 0.7 MPa·m^{1/2} as measured at 84 days from mixing.

The hardened glass ionomer cement may have a plane strain fracture toughness of at least 1 MPa·m^{1/2} as measured at 84 days from mixing.

The hardened glass ionomer cement may have any suitable toughness, G_{Ic}, as measured at 24 hours from mixing. As reported herein, the toughness was calculated according to the following equation:

$${G}_{IC} = \frac{{K}_{I c}^{2}}{E}$$

wherein K_{Ic} is the plane strain fracture toughness and E is Young's modulus.

All values for the toughness reported herein were measured this way.

The hardened glass ionomer cement may have a toughness of at least 70 J/m², such as at least 80 J/m², such as at least 90 J/m², such as at least 100 J/m², such as at least 110 J/m², such as at least 120 J/m², such as at least 130 J/m², such as at least 140 J/m², such as at least 150 J/m², such as at least 200 J/m², such as at least 250 J/m², such as at least 300 J/m², such as at least 350 J/m², such as at least 400 J/m², or even at least 450 J/m² as measured at 24 hours from mixing.

The hardened glass ionomer cement may have a toughness of at least 70 J/m² as measured at 24 hours from mixing.

The hardened glass ionomer cement may have a toughness of at least 100 J/m 2 as measured at 24 hours from mixing.

The hardened glass ionomer cement may have any suitable toughness as measured at 28 days from mixing. The hardened glass ionomer cement may have a toughness of at least 50 J/m², such as at least 55 J/m², such as at least 60 J/m², such as at least 65 J/m², such as at least J/m², such as at least 80 J/m², such as at least 90 J/m², such as at least 100 J/m², such as at least 110 J/m², such as at least 120 J/m², such as at least 130 J/m², such as at least 140 J/m², such as at least 150 J/m², such as at least 200 J/m², such as at least 250 J/m², such as at least 300 J/m², or even at least 350 J/m² as measured at 28 days from mixing.

The hardened glass ionomer cement may have a toughness of at least 50 J/m² as measured at 28 days from mixing.

The hardened glass ionomer cement may have a toughness of at least 65 J/m² as measured at 28 days from mixing.

The hardened glass ionomer cement may have any suitable toughness as measured at 84 days from mixing. The hardened glass ionomer cement may have a toughness of at least 40 J/m², such as at least 45, such as at least 50 J/m², such as at least 55 J/m², such as at least 60 J/m², such as at least 65 J/m², such as at least 70 J/m², such as at least 80 J/m², such as at least J/m², such as at least 100 J/m², such as at least 110 J/m², such as at least 120 J/m², such as at least 130 J/m², such as at least 140 J/m², such as at least 150 J/m², such as at least 200 J/m², or even at least 250 J/m² as measured at 84 days from mixing.

The hardened glass ionomer cement may have a toughness of at least 45 J/m² as measured at 84 days from mixing.

The hardened glass ionomer cement may have any suitable Young's modulus as measured at 24 hours from mixing. As reported herein, the Young's modulus was determined according to ASTM D790-17 (Flexural Tests for Plastics) as follows: after testing the plane strain fracture toughness, the broken halves of the specimen were first sanded down using 180 grit paper and then finished with a 1200 grit paper to ensure a good surface finish. The specimens were sanded down to approximately 3 mm×10 mm×65 mm (t×w×I). They were then tested at a constant crosshead speed of 1 mm/min in a Universal Tensile Tester (H10KS Tinius Olsen). The Young's modulus was calculated according to the following equation:

$$E = \frac{{s}^{3} ( \frac{P}{δ} )}{4 b {t}^{3}}$$

wherein s is the span, P is the peak load, 6 is the deflection at the centre of the specimen, b is the breadth of the specimen and t is the thickness of the specimen. All values for Young's modulus reported herein were measured this way.

The hardened glass ionomer cement may have a Young's modulus of at least 0.5, such as at least 1 GPa, such as at least 2 GPa, such as at least 3 GPa, such as at least 4 GPa, such as at least 5 GPa, such as at least 6 GPa, such as at least 7 GPa, or even at least 8 GPa as measured at 24 hours from mixing.

The hardened glass ionomer cement may have any suitable Young's modulus as measured at 28 days from mixing. The hardened glass ionomer cement may have a Young's modulus of at least 7 GPa, such as at least 8 GPa, such as at least 9 GPa, or even at least 10 GPa as measured at 28 days from mixing.

The hardened glass ionomer cement may have any suitable Young's modulus as measured at 84 days from mixing. The hardened glass ionomer cement may have a Young's modulus of at least 7 GPa, such as at least 8 GPa, such as at least 9 GPa, such as at least 10 GPa, or even at least 11 GPa as measured at 84 days from mixing.

The glass ionomer cement may have any suitable flexural strength, Of, as measured at 24 hours from mixing. As reported herein the flexural strength was determined according to the same method as outlined above for the measurement of Young's modulus, with the exception that the flexural strength was calculated according to the following equation:

$${σ}_{f} = \frac{3 P s}{2 b {t}^{2}}$$

wherein P is the peak load, s is the span, b is the breadth of the specimen and t is the thickness of the specimen. All values for the flexural strength reported herein were measured this way.

The hardened glass ionomer cement may have a flexural strength of at least 20 MPa, such as at least 25 MPa, such as at least 30 MPa, such as at least 35 MPa, such as at least 40 MPa, or even at least 55 MPa as measured at 24 hours from mixing.

Suitably, the hardened glass ionomer cement may have a flexural strength of at least 25 MPa as measured at 24 hours from mixing.

The glass ionomer cement may have any suitable flexural strength as measured at 28 days from mixing. The hardened glass ionomer cement may have a flexural strength of at least 20 MPa, such as at least 25 MPa, such as at least 30 MPa, such as at least 35 MPa, such as at least 40 MPa, or even at least 55 MPa as measured at 28 days from mixing.

Suitably, the hardened glass ionomer cement may have a flexural strength of at least 25 MPa as measured at 28 days from mixing.

Suitably, the hardened glass ionomer cement may have a flexural strength of at least 30 MPa as measured at 28 days from mixing.

The glass ionomer cement may have any suitable flexural strength as measured at 84 days from mixing. The hardened glass ionomer cement may have a flexural strength of at least 20 MPa, such as at least 25 MPa, such as at least 30 MPa, or even at least 35 MPa as measured at 28 days from mixing.

Suitably, the hardened glass ionomer cement may have a flexural strength of at least 25 MPa as measured at 84 days from mixing.

Suitably, the hardened glass ionomer cement may have a flexural strength of at least 55 MPa as measured at 84 days from mixing.

Advantageously, the hardened glass ionomer cements of the present invention may have an improved fracture toughness, toughness, Young's modulus and/or flexural modulus than would typically be expected even after, for example, 84 days.

There is no particular temperature limitation on the use of the present invention. Suitably, however, it is used at temperatures acceptable to the operator i.e. temperatures found during normal working conditions that may be encountered indoors or outdoors by the operator, for example 5-40° C. and atmospheric pressure and/or applied syringe pressure.

For medical applications, the composition is suitably biocompatible and in particular suitably hardens to a solid glass ionomer cement that is biocompatible in situ. Thus, the two part hardenable composition of the present invention may find particularly advantageous utility as a medical implant material.

Thus, according to a fourth aspect of the present invention there is provided a two part hardenable composition according to the first aspect of the present invention for use in the treatment of human or animal bone.

Suitable features of the fourth aspect of the present invention are as defined herein in relation to the first, second and/or third aspects of the present invention.

The two part hardenable compositions of the present invention may also find particularly advantageous utility as a dental implant material.

According to a fifth aspect of the present invention there is provided a two part hardenable composition according to the first aspect of the present invention for use in dentistry.

Suitable features of the fifth aspect of the present invention are as defined herein in relation to the first, second, third and/or fourth aspects of the present invention.

As used herein, unless otherwise expressly specified, all numbers such as those expressing values, ranges, amounts or percentages may be read as if prefaced by the word “about”, even if the term does not expressly appear. Also, the recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

Singular encompasses plural and vice versa. For example, although reference is made herein to “a” basic glass, “a” water-miscible additive, “an” acid-functional polymer, and the like, one or more of each of these and any other components can be used. As used herein, the term “polymer” refers to oligomers and both homopolymers and copolymers, and the prefix “poly” refers to two or more. Including, for example and like terms means including for example but not limited to.

The terms “comprising”, “comprises” and “comprised of” as used herein are synonymous with “including”, “includes” or “containing”, “contains”, and are inclusive or open-ended and do not exclude additional, non-recited members, elements or method steps. Additionally, although the present invention has been described in terms of “comprising”, the coating compositions detailed herein may also be described as “consisting essentially of” or “consisting of”.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination. As used herein, the term “and combinations thereof” when used in a list of two or more items, means that any one of the listed items can be employed by itself or any combination of two or more of the listed items can be employed. For example, if a list is described as comprising group A, B, C and combinations thereof, the list can comprise A alone; B alone; C alone; A and B in combination; A and C in combination, B and C in combination; or A, B, and C in combination.

All of the features contained herein may be combined with any of the above aspects in any combination.

For a better understanding of the invention, and to show how embodiments of the same may be carried into effect, reference will now be made, by way of example, to the following examples.

### EXAMPLES

### Examples 1(a) to (j)

Glass ionomer cements were prepared by mixing, at room temperature, a two part hardenable composition comprising a powder first part containing 1 gram (g) of a representative basic glass of the following composition: 1.5 SiO₂, Al₂O₃, 0.5 P₂O⁵, 0.5 CaO, 0.5 SrO, 0.66 CaF₂ with a liquid second part containing 0.2 g BU090, a polyacrylic acid commercially available from Advanced Healthcare, 0.2 g water containing 10% w/v of tartaric acid and a reactive additive in accordance with Table 1. Examples 1(a) to (e) contained poly(ethylene glycol) diglycidyl ether (PEGDGE) and examples 1(f) to (j) contained 1,4-butanediol diglycidyl ether (BDE) in the amounts given in Table 1, wherein mole % is based on the number of moles of the repeat unit of the acid-functional polymer, i.e. acrylic acid.

### Comparative Example 1

Comparative example 1 was prepared according to examples 1(a) to (j) above with the exception that no reactive additive was added.

The working time of examples 1(a) to (j) and comparative example 1 was measured and recorded. The results are provided in table 1.

TABLE 1
Working time of examples 1(a) to (j) and comparative example 1

| | | Amount of | |
|---|---|---|---|
| | | additive/ | Working |
| Example | Additive | mole % | time/mins |
| Comparative | — | — | 5.8 |
| example 1 | | | |
| 1(a) | PEGDGE | 0.25 | 6.5 |
| 1(b) | PEGDGE | 0.5 | 6.5 |
| 1(c) | PEGDGE | 1 | 7.5 |
| 1(d) | PEGDGE | 2 | 9 |
| 1(e) | PEGDGE | 3 | 12 |
| 1(f) | BDE | 0.25 | 6 |
| 1(g) | BDE | 0.5 | 6 |
| 1(h) | BDE | 1 | 7.5 |
| 1(i) | BDE | 2 | 7.5 |
| 1(j) | BDE | 3 | 9.4 |

The results show that the inventive examples containing a reactive additive have longer working times compared to the comparative example.

### Examples 2(a) to (k)

Glass ionomer cements were prepared by mixing, at room temperature, a two part hardenable composition comprising a powder first part containing 1 gram (g) of a basic glass of the following composition: 1.5 SiO₂, Al₂O₃, 0.5 P₂O₅, 0.5 CaO, 0.5 SrO, 0.66 CaF₂ with a liquid second part containing 0.2 g BU090, a polyacrylic acid commercially available from Advanced Healthcare, 0.2 g water containing 10% w/v of tartaric acid and a dispersion additive in accordance with Table 2. Examples 2(a) to (e) contained triethyl citrate (TEC) and examples 2(f) to (k) contained polysorbate 80 in the amounts given in Table 2, wherein mole % is based on the number of moles of the repeat unit of the acid-functional polymer, i.e. acrylic acid. The glass volume fraction of the glass ionomer cements was 0.50.

The working time of examples 2(a) to (k) and comparative example 1 was measured and recorded. The results are provided in table 2.

### Examples 3(a) to (w)

Examples 3(a) to (n) were prepared in accordance with example 2(a) above with the following exceptions: Glascol E11, a polyacrylic acid commercially available from Allied Colloids, was used instead of BU090, the concentration of the acid-functional polymer was 47% instead of 50% (based on the total weight of the acid-functional polymer and water) and the glass volume fraction (GVf) was adjusted according to Table 3.

Similarly, Examples 3(o) to (w) were prepared in accordance with example 2(f) above with the following exceptions: the acid-functional polymer E11, a polyacrylic acid commercially available from Allied Colloids, was used instead of BU090, the concentration of the acid-functional polymer was 47% instead of 50% (based on the total weight of the acid-functional polymer and water) and the glass volume fraction (GVf), i.e. the volume of the basic glass divided by the volume of all constituents in the two part hardenable composition, was adjusted according to Table 3.

### Comparative Example 2

Comparative example 2 was prepared according to examples 3(a) to (w) above with the exception that no reactive additive was added.

The working time of examples 3(a) to (w) and comparative example 2 was measured and recorded. The results are provided in table 3.

TABLE 2
Working time of examples 2(a) to (k) and comparative example 1

| | | Amount of | | |
|---|---|---|---|---|
| | | additive/ | | Working |
| Example | Additive | mole % | GVf | time/min |
| Comparative | — | — | 0.50 | 6 |
| example 1 | | | | |
| 2(a) | TEC | 2.6 | 0.50 | 10 |
| 2(b) | TEC | 5.21 | 0.50 | 16 |
| 2(c) | TEC | 7.82 | 0.50 | 17 |
| 2(d) | TEC | 10.42 | 0.50 | 20.5 |
| 2(e) | TEC | 13.03 | 0.50 | 22.5 |
| 2(f) | Polysorbate 80 | 0.55 | 0.50 | 9 |
| 2(g) | Polysorbate 80 | 1.1 | 0.50 | 11.5 |
| 2(h) | Polysorbate 80 | 1.65 | 0.50 | 13 |
| 2(i) | Polysorbate 80 | 2.61 | 0.50 | 17 |
| 2(j) | Polysorbate 80 | 7.82 | 0.50 | >30 |
| 2(k) | Polysorbate 80 | 13.03 | 0.50 | >30 |

TABLE 3
Working time of examples 3(a) to (w) and comparative example 2

| | | Amount of | | |
|---|---|---|---|---|
| | | additive/ | | Working |
| | Additive | mole % | GVf | time/min |
| Comparative | — | — | 0.49 | 3 |
| example 2 | | | | |
| 3(a) | TEC | 5.21 | 0.49 | 6 |
| 3(b) | TEC | 7.82 | 0.49 | 7 |
| 3(c) | TEC | 10.42 | 0.49 | 7.25 |
| 3(d) | TEC | 12 | 0.49 | 10.5 |
| 3(e) | TEC | 5.21 | 0.50 | 6 |
| 3(f) | TEC | 7.82 | 0.50 | 6.5 |
| 3(g) | TEC | 7.82 | 0.51 | 6 |
| 3(h) | TEC | 10.42 | 0.51 | 6.75 |
| 3(i) | TEC | 12 | 0.51 | 8.5 |
| 3(j) | TEC | 10.42 | 0.53 | 6 |
| 3(k) | TEC | 12 | 0.53 | 7.5 |
| 3(l) | TEC | 10.42 | 0.55 | 5.5 |
| 3(m) | TEC | 12 | 0.55 | 6.5 |
| 3(n) | TEC | 12 | 0.57 | 5.75 |
| 3(o) | Polysorbate 80 | 0.55 | 0.49 | 4.25 |
| 3(p) | Polysorbate 80 | 1.65 | 0.49 | 5.5 |
| 3(q) | Polysorbate 80 | 2.61 | 0.49 | 6.5 |
| 3(r) | Polysorbate 80 | 0.55 | 0.51 | 3.25 |
| 3(s) | Polysorbate 80 | 1.65 | 0.51 | 4.25 |
| 3(t) | Polysorbate 80 | 2.61 | 0.51 | 6 |
| 3(u) | Polysorbate 80 | 2.61 | 0.53 | 5 |
| 3(v) | Polysorbate 80 | 2.61 | 0.55 | 4.5 |
| 3(w) | Polysorbate 80 | 2.61 | 0.57 | 4.75 |

The results show that the inventive examples containing a dispersion additive have longer working times compared to the comparative example. The results also show that the glass volume fraction (GVf) can be increased in the presence of the additives according to the invention without adversely affecting the working time.

The mechanical properties of the glass ionomer cements were also tested according to the following test methods.

Plain strain fracture toughness: the plane strain fracture toughness, K_{Ic}, of the glass ionomer cements was determined by a double torsion (DT) test as outlined in de Barra E. and Hill R. “Influence of Alkali Metal Ions on the Fracture Properties of Glass Polyalkenoate Cements” Bio materials 30 495-502(1998). The DT specimens of 3 mm×65 mm×25 mm (t×I×w) were produced using stainless steel moulds. The cement mixture was placed in these moulds and pressed between two plates using a G-clamp. A silicon-based mould release agent was used on the inner edges of the mould for ease of removal. They were then stored in a forced air oven pre-set to 37° C.±2° C. for one hour. After this time, they were removed from the mould and placed in half litre containers filled with deionised water at 37° C.±2° C. until they were ready to be tested. For testing, a groove of 1 mm×1 mm was cut down the centre of the specimen as well as a sharp groove at the end of the specimen. Testing was carried out on a Universal Tensile Tester (H10KS Tinius Olsen) in a water bath at 37° C.±2° C. The specimen was loaded onto two parallel rollers 3 mm in diameter and spaced 20 mm apart. A constant crosshead speed of 0.1 mm/min was then applied to the end of the specimen which had the sharp groove with two 3 mm ball bearings spaced 10 mm apart (such that the loaded end of the specimen was subjected to a four-point bend). The plane strain fracture toughness, K_{Ic}, was calculated according to the following equation:

$${K}_{Ic} = {P}_{C} {{W}_{m} ( \frac{3 ( 1 + v )}{W {t}^{3} {t}_{n}} )}^{\frac{1}{2}}$$

wherein P_{c} is the critical load required to fracture the specimen, Wₘ is the moment arm, W is the specimen width, t is the specimen thickness, tₙ is the web thickness of the groove and v is Poisson's ratio (assumed to be 0.33). The plane strain fracture toughness was measured in this way at 24 hours, 28 days and 84 days.

Toughness: the toughness, G_{Ic}, of the glass ionomer cements was calculated according to the following equation:

$${G}_{IC} = \frac{{K}_{I c}^{2}}{E}$$

wherein K_{Ic} is the plane strain fracture toughness and E is Young's modulus.

Flexural strength: the flexural strength of the glass ionomer cements was tested according to the same method as for the measurement of Young's modulus, with the exception that the flexural strength was calculated according to the following equation:

$${σ}_{f} = \frac{3 P s}{2 b {t}^{2}}$$

wherein P is the peak load, s is the span, b is the breadth of the specimen and t is the thickness of the specimen. All values for the flexural strength reported herein were measured this way.

The results are shown in Table 4.

TABLE 4
Results

| | | | | | |
|---|---|---|---|---|---|
| Comparative | Example | Example | Example | Example | Example |
| example 1 | 1(f) | 1(g) | 1(h) | 1(i) | 1(j) |

Plane Strain Fracture Toughness/MPa · m^{1/2}

| | | | | | | | | | | | | |
|---|---|---|---|---|---|---|---|---|---|---|---|---|
| 24 Hours | 0.84 | (0.08) | 0.93 | (0.07) | 0.75 | (0.04) | 0.87 | (0.08) | 1.49 | (0.05) | 1.47 | (0.09) |
| (S.D.) | | | | | | | | | | | | |
| 28 days | 0.69 | (0.06) | 0.82 | (0.04) | 0.87 | (0.05) | 0.91 | (0.05) | 1.28 | (0.12) | 1.65 | (0.08) |
| (S.D.) | | | | | | | | | | | | |
| 84 days | 0.68 | (0.05) | 0.72 | (0.05) | 0.86 | (0.09) | 0.8 | (0.07) | 1.13 | (0.08) | 1.37 | (0.1) |
| (S.D.) | | | | | | | | | | | | |

Toughness/J/m²

| | | | | | | | | | | | | |
|---|---|---|---|---|---|---|---|---|---|---|---|---|
| 24 Hours | 97.8 | (6.89) | 144.25 | (10.11) | 70.25 | (2.58) | 98.35 | (26.57) | 391.23 | (12.06) | 489.6 | (29.44) |
| (S.D.) | | | | | | | | | | | | |
| 28 days | 44.01 | (7.28) | 68.19 | (2.38) | 73.28 | (3.15) | 82.61 | (4.26) | 176.11 | (54.51) | 353.37 | (17.66) |
| (S.D.) | | | | | | | | | | | | |
| 84 days | 39.91 | (3.8) | 47.37 | (3.76) | 70.36 | (12.95) | 62.62 | (7.56) | 149.2 | (16.53) | 264.67 | (17) |
| (S.D.) | | | | | | | | | | | | |

Flexural Strength/MPa

| | | | | | | | | | | | | |
|---|---|---|---|---|---|---|---|---|---|---|---|---|
| 24 Hours | 24.92 | (2.24) | 28.86 | (3.36) | 33 | (3.14) | 39 | (3.11) | 47.35 | (2.58) | 43.42 | (1.67) |
| (S.D.) | | | | | | | | | | | | |
| 28 days | 29.11 | (1.81) | 27.72 | (2.68) | 31.49 | (2.35) | 31.7 | (2.37) | 44.51 | (2.43) | 46.64 | (2.86) |
| (S.D.) | | | | | | | | | | | | |
| 84 days | 26.94 | (1.16) | 28.11 | (2.6) | 30.73 | (3.16) | 31.36 | (2.96) | 35.4 | (2.65) | 38.15 | (3.47) |
| (S.D.) | | | | | | | | | | | | |

The results show that the mechanical properties of the inventive examples are as good as, or better than, those of the comparative examples as measured at each of 24 hours, 28 days and 84 days.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A two part hardenable composition comprising a powder first part and a liquid second part, the parts being operable to form a glass ionomer cement which hardens to a solid mass upon mixing of the parts together, the composition comprising an inorganic glass and/or salt in the powder first part, an aqueous carrier in the liquid second part and an acid-functional polymer in the powder first part and/or the liquid second part;
wherein the composition further comprises an at least partially water-miscible additive in the first and/or second parts, the additive being operable to extend the working time of the glass ionomer cement upon mixing of the first and or second parts; and
wherein the composition is operable to form a glass ionomer cement which, once hardened to a solid mass, has a plane strain fracture toughness of at least 0.7 MPa·m^{1/2} as measured at 24 hours from mixing.

2. A two part hardenable composition according to claim 1, wherein the additive comprises a reactive additive operable to form a covalent linkage with one or more polymer chains of the acid-functional polymer and/or a dispersion additive operable to become dispersed within the polysalt matrix of the hardened glass ionomer cement.

3. A two part hardenable composition according to claim 2, wherein the reactive additive comprises at least one functional group operable to react with acid-functionality on the acid-functional polymer.

4. A two part hardenable composition according to claim 3, wherein the reactive additive comprises at least one oxirane, amine, hydroxyl and/or carbodiimide group.

5. A two part hardenable composition according to claim 4, wherein the reactive additive comprises neopentyl glycol diglycidyl ether, glycerol diglycidyl ether, 1,4-butanediol diglycidyl ether, N-(3-dimethylaminopropyl)-N′-ethylcarbodiimide, 1,3-butadiene diepoxide, adipic acid dihydrazide, polyethylenimine, ethylenediamine, poly(ethylene glycol) diglycidyl ether, polyproplene glycol diglycidyl ether, glutaraldehyde or combinations thereof.

6. A two part hardenable composition according to claim 2, wherein the dispersion additive comprises triethyl citrate, tributyl citrate, acetyl tributyl citrate, polysorbate 80, polyethylene glycol, propylene glycol, 1,2 epoxybutane, 1,2-epoxydodecane, 1,2-epoxypentane, 1,2-epoxytetradecane, glycidyl hexadecyl ether, glycidyl isopropyl ether, octyl glycidyl ether, decyl glycidyl ether or combinations thereof.

7. A two part hardenable composition according to claim 1, wherein the two part hardenable composition comprises from 0.05 to 20 mole % additive based on the number of moles of the repeat (or monomer) unit of the acid-functional polymer.

8. A two part hardenable composition according to claim 1, wherein the inorganic salt and/or glass comprises a basic glass.

9. A two part hardenable composition according to claim 1, wherein the two part hardenable composition has a glass volume fraction from 40 to 60%. (currently amended) A two part hardenable composition according to claim 1, wherein the aqueous carrier comprises 100 vol % water based on the total volume of the aqueous carrier.

11. A two part hardenable composition according to claim 1, wherein the acid-functional polymer comprises a homopolymer and/or copolymer of acrylic acid.

12. A two part hardenable composition according to claim 1, wherein the acid-functional polymer has an acid number of at least 10 mg KOH/g.

13. A two part hardenable composition according to claim 1, wherein the acid-functional polymer has an Mn from 10,000 to 150,000 Da.

14. A two part hardenable composition according to claim 1, wherein the composition is operable to form a glass ionomer cement which, once hardened to a solid mass, has a plane strain fracture toughness of at least 0.7 MPa·m^{1/2} as measured at 24 hours from mixing.

15. A two part hardenable composition according to claim 1, wherein the composition is operable to form a glass ionomer cement which, once hardened to a solid mass, has a toughness of at least 70 J/m² measured at 24 hours from mixing.

16. A two part hardenable composition according to claim 1, wherein the composition is operable to form a glass ionomer cement which, once hardened to a solid mass, has a flexural strength of at least 25 MPa as measured at 24 hours from mixing.

17. A method of producing a glass ionomer cement from a two part hardenable composition according to claim 1, the method comprising the step of mixing the powder first part and liquid second part.

18. A solid glass ionomer cement produced from mixing the powder first part and liquid second part of the two part hardenable composition according to claim 1.

19. (canceled)

20. (canceled)
